(19) United States
(12) Patent Application Publication
(10) Pub. No.: US 2013/0217776 A1
(43) Pub. Date: Aug. 22, 2013

(54) Combination of a Serotonin Reuptake Inhibitor and Agomelatine

(71) Applicant: Sandra Willigers, Bagsvaerd (DK)

(72) Inventor: Sandra Willigers, Bagsvaerd (DK)

(73) Assignee: H. Lundbeck A/S, Valby-Copenhagen (DK)

(21) Appl. No.: 13/552,699

(22) Filed: Jul. 19, 2012

Related U.S. Application Data
(63) Continuation of application No. 12/764,348, filed on Apr. 21, 2010, now abandoned.
Application No. 12/764,348 is a continuation of application No. 10/558,494, filed on Feb. 9, 2006, now abandoned.
(60) Provisional application No. 60/485,479, filed on Jul. 7, 2003.

(30) Foreign Application Priority Data
Jul. 4, 2003 (DK) PA200301031

(51) Int. Cl. A61K 31/165; A61K 45/06

(52) U.S. Cl. CPC A61K 31/165; A61K 45/06
USPC 514/630

(57) ABSTRACT

The present invention relates to the use of a combination of agomelatine and a serotonin reuptake inhibitor (SRIs), or any other compound, which causes an elevation in the level of extracellular serotonin, for the treatment of depression and other affective disorders.

The present invention relates to the use of a combination of agomelatine and a serotonin reuptake inhibitor (SRI), or any other compound, which causes an elevation in the level of extracellular serotonin, for the treatment of depression and other affective disorders.

### BACKGROUND

Selective serotonin reuptake inhibitors (hereinafter referred to as SSRIs) have become to first choice therapeutics in the treatment of depression, certain forms of anxiety and social phobias, because they are effective, well tolerated and have a favourable safety profile compared to the classic tricyclic antidepressants.

However. clinical studies on depression and anxiety disorders indicate that non-response to SSRIs is substantial, up to 30%. Another, often neglected, factor in antidepressant treatment is compliance, which has a rather profound effect on the patient's motivation to continue pharmacotherapy.

First of all, there is the delay in therapeutic effect of SSRIs. Sometimes symptoms even worsen during the First weeks of treatment. Secondly, sexual dysfunction is a side effect common to all SSRIs. Without addressing these problems real progress in the pharmacotherapy of depression and anxiety disorders is not likely to happen.

In order to cope with non-response, psychiatrists sometimes make use of augmentation strategies. Augmentation of antidepressant therapy may he accomplished through the co-administration of mood stabilizers such as lithium carbonate or triiodothyronin or by the use of electroshock.

In 1993, an augmentation strategy with pindolol was described by Artigas et al. in Trends Pharmacol. Sci. 1993. 14, p 262-263. Artigas' idea is based on intracerebral microdialysis experiments in animals. In fact, later neurochemical studies built on the desensitization hypothesis by Blier and co-workers stated that the delay in therapeutic effect of antidepressants is related to a gradual desensitization of 5-HT autoreceptors (Slier et al. J. Clin. Psycipharmacol. 1987, 7 suppl. 6, 24S-35S). A key point in their hypothesis is that the effects of SSRIs on the release-controlling somatodendritic autoreceptors (5-HT_{1A}) limit the release of 5-HT in terminal areas and thus the effect of 5-HT uptake inhibition in those regions. This is supported by microdialysis experiments in rats, showing that the increase in extracellular 5-HT elicited by a single dose of an SSR1 is augmented by co-administration of a 5-HT_{1A} autoreceptor antagonist (Invernizzi et al. Brain Res. 1992, 584, p 322-324 and Hjorth, S., J. Neurochem, 1993, 60, p 776-779).

The effect of combined administration of a compound that inhibits serotonin reuptake and a 5-HT_{1A} receptor antagonist has been evaluated in several studies (Innis, R. B. et al. Eur. J. Pharmacol. 1987. 143, p. 1095-204 and Gartside, S. E., Br. J. Pharmacol. 1995, 115. p 1064-1070, Blier. P. et al. Trends in Pharmacol. Science 1994, 15, 220). In these studies it was found that 5-HT_{1A} receptor antagonists would abolish the initial brake on 5-HT neurotransmission induced by the serotonin reuptake inhibitors and thus produce an immediate boost of 5-HT transmission and a rapid onset of therapeutic action.

Several patent applications have been filed which cover the use of a combination of a 5-HT_{1A} antagonist and a serotonin reuptake inhibitor for the treatment of depression (see e.g. EP-A2-687 472 and EP-A2-714 663).

Another approach to increase terminal 5-HT would be through blockade of the 5-HT_{1B} autoreceptor. Microdialysis experiments in rats have indeed shown that increase of hippocampal 5-HT by citalopram is potentiated by GMC 2-29, an experimental 5-HT_{1B} receptor antagonist.

Several patent applications covering the combination of an SSRI and a 5-HT_{1B} antagonist or partial agonist have also been filed (WO 97/28141, WO 96/03400, EP-A-701819 and WO 99/13877).

It has now surprisingly been found that agomelatine having the general formula

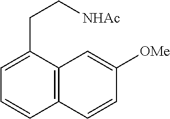

or pharmaceutically acceptable salts thereof may be used to augment and provide faster onset of the therapeutic effect of serotonin reuptake inhibitors.

### THE INVENTION

The present invention thus provides:

The present invention relates to the use of agomclatine or a pharmaceutically acceptable salt thereof for the preparation of a pharmaceutical composition to be used in combination with a serotonin reuptake inhibitor or any other compound, which causes an elevation in the level of extracellular serotonin.

In particular, the present invention relates to the use of agomclatine or a pharmaceutically acceptable salt thereof for the preparation of a pharmaceutical composition useful for augmenting and/or providing faster onset of the therapeutic effect of a serotonin reuptake inhibitor or any other compound, which causes an elevation in the level of extracellular serotonin.

More particularly, the present invention relates to the use as above, of agomelatine, or a pharmaceutically acceptable salt thereof, for the treatment of depression, anxiety disorders and other affective disorders, eating disorders such as bulimia, anorexia and obesity, phobias, dysthymia, premenstrual syndrome, cognitive disorders, impulse control disorders, attention deficit hyperactivity disorder and drug abuse, in particular depression with a serotonin reuptake inhibitor or any other compound, which causes an elevation in the level of extracellular serotonin.

The anxiety disorders mentioned above include general anxiety disorder, panic anxiety, obsessive compulsive disorder, acute stress disorder. post trauma stress disorder or social anxiety disorder.

As used herein, the term augmenting covers improving the therapeutic effect and/or potentiating the therapeutic effect of an SRI or a compound which causes an elevation in the level of extracellular 5-HT.

In a further embodiment, the invention relates to the use of agomelatine or a pharmaceutically acceptable salt thereof and a compound, which is a serotonin reuptake inhibitor, or a compound, which causes an elevation in the level of extracellular serotonin, for the preparation of a pharmaceutical composition or kit for the treatment of diseases or disorders responsive to the therapeutic effect of a serotonin reuptake inhibitor, or any other compound, which causes an elevation in the level of extracellular serotonin.

The diseases responsive to a serotonin reuptake inhibitor include depression, anxiety disorders and other affective disorders, eating disorders such as bulimia, anorexia and obesity, phobias, dysthymia, premenstrual syndrome, cognitive disorders, impulse control disorders, attention deficit hyperactivity disorder and drug abuse, in particular depression.

The term anxiety disorders is as defined above.

In one embodiment, the present invention relates to the use of agomelatine or a pharmaceutically acceptable salt thereof for the preparation of a pharmaceutical composition as above, which is adapted for simultaneous administration of the active ingredients. In particular, such pharmaceutical compositions may contain the active ingredients within the same unit dosage form, e.g. in the same tablet or capsule. Such unit dosage forms may contain the active ingredients as a homogenous mixture or in separate compartments of the unit dosage form.

In another embodiment, the present invention relates to the use of agomelatine or a pharmaceutically acceptable salt thereof for the preparation of a pharmaceutical composition or kit as above, which is adapted for sequential administration of the active ingredients. In particular, such pharmaceutical compositions may contain the active ingredients in discrete unit dosage forms, e.g. discrete tablets or capsules containing either of the active ingredients. These discrete unit dosage forms may be contained in the same container or package, e.g. a blister pack.

As used herein the term kit means a pharmaceutical composition containing each of the active ingredients, but in discrete unit dosage forms.

The invention also relates to a pharmaceutical composition or kit comprising agomelatine or a pharmaceutically acceptable salt thereof and a compound, which is a serotonin reuptake inhibitor, or any other compound, which causes an elevation in extracellular 5-HT, and optionally pharmaceutically acceptable carriers or diluents.

The pharmaceutical composition or kit of the invention may he adapted for simultaneous administration of the active ingredients or for sequential administration of the active ingredients, as described above.

Finally, the present invention relates to a method for the treatment of diseases or disorders responsive to a serotonin reuptake inhibitor or any other compound, which causes an elevation in the level of extracellular serotonin, comprising administering agomelatine or a pharmaceutically acceptable salt thereof and a serotonin reuptake inhibitor, or a compound, which causes an elevation in the level extracellular serotonin, to an individual in need thereof.

In particular, the present invention relates to a method for augmenting and/or providing faster onset of the therapeutic effect of a serotonin reuptake inhibitor or any other compound, which causes an elevation in the level extracellular serotonin, comprising administering agomelatine or a pharmaceutically acceptable salt thereof to an individual to be treated with or undergoing treatment with the serotonin reuptake inhibitor or any other compound, which causes an elevation in the level of extracellular serotonin. The individuals, which may benefit from treatment with a combination as above, may suffer from depression, anxiety disorders and other of disorders, eating disorders such as bulimia, anorexia and obesity, phobias, premenstrual syndrome, dysthymia, to cognitive disorders, impulse control disorders, attention deficit hyperactivity disorder and drug abuse, in particular depression.

As mentioned above, anxiety disorder includes general anxiety disorder, panic anxiety, obsessive compulsive disorder, acute stress disorder, post trauma stress disorder or social anxiety disorder.

Agomelatine and the serotonin reuptake inhibitor may be administered simultaneously as described above.

Alternatively, the active ingredients may he administered sequentially, e.g. in two discrete unit dosage forms as described above.

Agomelatine and pharmaceutically acceptable salts thereof having the general formula

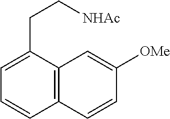

are covered by EP-B1-447285. The compounds claimed therein are described as having affinity for the melatonin receptor.

It has now, surprisingly, been found that co-administration of agomelatine and a serotonin reuptake inhibitor produces a significant increase in the level of serotonin in terminal areas, as measured in microdialysis experiments, compared to the administration of the serotonin reuptake inhibitor alone. Administration of agomelatine alone causes no increase in serotonin levels as measured in microdialysis experiments.

As mentioned above, serotonin reuptake inhibitors show delayed onset of action. Even in responders to SSRIs, several weeks of treatment are necessary to achieve a relief in symptoms. Agomclatine may provide fast onset of therapeutic effect of serotonin reuptake inhibitors.

The use of a combination of agomelatine and a serotonin reuptake inhibitor may greatly reduce the amount of serotonin reuptake inhibitor necessary to treat depression and other affective disorders and may thus reduce the side effects caused by the serotonin reuptake inhibitor. In particular, the combination of a reduced amount of SRI and agomelatine may reduce the risk of SSRI-induced sexual dysfunction and sleep disturbances.

Co-administration of agomelatine and a serotonin reuptake inhibitor may also be useful for the treatment of refractory depression, i.e. depression, which cannot be treated appropriately by administration of a serotonin reuptake inhibitor alone. Typically, agomelatine may be used as add-on therapy for the augmentation of the response to SRIs in patients where at least 40-60% reduction in symptoms has not been achieved during the first 6 weeks of treatment with an SRI.

Many antidepressants with serotonin reuptake inhibiting effect have been described in the literature. Any pharmacologically active compound which primarily or partly exert its therapeutic effect via inhibition of serotonin reuptake in the CNS, may benefit from augmentation with agomelatine.

The following list contains a number of serotonin reuptake inhibitors, which may benefit from augmentation with agomelatine: citalopram, escitalopram, fluoxetine, R-fluoxetine. sertraline, paroxetine, fluvoxamine, venlafaxine, duloxetine, dapoxetine, nefazodone, imipramine, imipramine N-oxide, desipramine, pirandamine, dazepinil, nefopam, befuraline, fezolamine, femoxetine, clomipramine, cianuimipramine, litoxetine, cericlamine, seproxetine, WY 27587, WY 27866, imeldine, ifoxetine, tiflucarbine, viqualine, milnacipran, hazinaprine, YM 922, S 33005, F 98214-TA, OPC 14523, alaproclate, cyanodothepine, trimipramine, quinupramine, dothiepin, amoxapine, nitroxazepine, McN 5652, McN 5707, Ol 77, Org 6582, Org 6997, Org 6906, amitriptyline, amitriptyline N-oxide, nortriptyline, CL 255.663, pirlindole, indatraline, LY 113.821, LY 214.281, CGP 6085 A, RU 25.591, napamezole, diclofensine, trazodone, EMD 68.843, BMY 42.569, NS 2389, sercloremine, nitroquipazine, ademethionine, sibutramine and clovoxamine. The compounds mentioned above may he used in the form of the base or a pharmaceutically acceptable acid addition salt thereof.

Other therapeutic compounds which,may benefit from augmentation with agomelatine include compounds, which causes an elevation in the extracellular level of 5-HT in the synaptic cleft, although they are not serotonin reuptake inhibitors. One such compound is tianeptine.

The above list of serotonin reuptake inhibitors and other compounds, which causes an increase in the extracellular level of serotonin, may not be construed as limiting.

SRIs which are particularly preferred according to the present invention include citalopram, escitalopram, fluoxetine, sertraline, paroxetine, fluvoxamine, venlafaxine, dapoxetine, nefazodone, imipramin, femoxetine and clomipramine.

The term selective serotonin reuptake inhibitor (SSRI) means an inhibitor of the monoamine transporters which has stronger inhibitory effect at the serotonin transporter than the dopamine and the noradrenaline transporters.

Selective serotonin reuptake inhibitors (SSRIs) arc among the most preferred serotonin reuptake inhibitors used according to the present invention. Particularly preferred SSRIs according to the invention are citalopram, escitalopram, fluoxetine, fluvoxamine, sertraline or paroxetine.

The active ingredients according to the invention, i.e. agomelatine and the SRI or a compound causing an increase in extracellular serotonin levels, may he used in the free base form or in the form of a pharmaceutically acceptable acid addition salt thereof, the latter being obtainable by reaction of the base form with an appropriate acid.

Citalopram is preferably used in the form of the hydrobromide or as the base, escitalopram in the form of the oxalate, fluoxetine, sertraline and paroxetine in the form of the hydrochloride and fluvoxamine in the form of the maleate.

As mentioned above, the combination of agomelatine with a serotonin reuptake inhibitor unexpectedly shows a synergistic effect on the central nervous system (CNS). As a consequence, combination therapy using agomelatine and lower doses of the serotonin reuptake inhibitor than normally used in monotherapy, may be effective, and side-effects associated with the larger amounts of serotonin reuptake inhibitor used in monotherapy may he reduced or prevented altogether.

Additionally, combination therapy with agomelatine using a normal dose of serotonin reuptake inhibitor has the advantage that an effective CNS effect may be obtained in the often large number of patients who do not respond to conventional monotherapy with SSRIs.

The amount of agomelatine used in combination therapy may range from about 0.1 to about 150 mg/day, particularly from about 0.1 to about 100 mg/day and more particularly from about 0.5 to about 50 mg/day and even more particularly from about 1 to about 5 mg/day.

Serotonin reuptake inhibitors, including the SSRIs specifically mentioned hereinabove, differ both in molecular weight and in activity. As a consequence, the amount of serotonin reuptake inhibitor used in combination therapy depends on the nature of said serotonin reuptake inhibitor. In one embodiment of the invention, the serotonin reuptake inhibitor or the compound causing an increase in the level of extracellular 5-HT, is administered at lower doses than required when the compound is used alone. In another embodiment, the serotonin reuptake inhibitor or the compound causing an increase in the level of extracellular 5-HT, is administered in normal doses.

To prepare the pharmaceutical compositions of this invention, an appropriate amount of the active ingredient(s), in salt form or base form, is combined in an intimate admixture with a pharmaceutically acceptable carrier, which can take a wide variety of forms depending on the form of preparation desired for administration. These pharmaceutical compositions are desirably in unitary dosage form suitable for administration orally, rectally, percutaneously or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the ease of powders, pills, capsules and tablets. Because of their ease in administration, tablets and capsules represent the most advantageous oral dosage unit form, in which case solid pharmaceutical carriers are obviously employed.

It is especially advantageous to formulate the aforementioned pharmaceutical compositions in dosage unit form for ease of administration and uniformity of dosage. As used in the specification and claims, unit dosage form refers to physically discrete units suitable as unitary dosages, each unit containing a predetermined quantity of active ingredient(s) calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Agomelatine may be administered before, during or after the administration of the serotonin reuptake inhibitor provided that the time between the administration of agomelatine and the administration of the serotonin reuptake inhibitor is such that ingredients are allowed to act synergistically on the CNS. When simultaneous administration of agomelatine and a serotonin reuptake inhibitor is envisaged, a composition containing both a serotonin reuptake inhibitor and agomelatine may be to particularly convenient. Or, agomelatine and the serotonin reuptake inhibitor may be administered separately in the form of suitable compositions. The compositions may be prepared as described hereinabove.

The present invention also comprises products containing agomelatine and a serotonin reuptake inhibitor as a combination preparation for simultaneous, separate or sequential use in psychiatric drug therapy. Such products may comprise, for example, a kit comprising discrete unit dosage forms containing agomelatine and discrete unit dosage forms containing a serotonin reuptake inhibitor, all contained in the same container or pack, e.g. a blister pack.

The above mentioned preparations for simultaneous or sequential administration may instead of a serotonin reuptake inhibitor contain another compound causing an elevation in the level of extracellular 5-HT.

1. The use of agomelatine or a pharmaceutically acceptable salt thereof for the preparation of a pharmaceutical composition to be used in combination with a serotonin reuptake inhibitor inhibitor or any other compound, which causes an elevation in the level of extracellular serotonin.

2-43. (canceled)
